(19) United States
(12) Patent Application Publication
(10) Pub. No.: US 2026/0120282 A1
(43) Pub. Date: Apr. 30, 2026

(54) METHOD, APPARATUS AND PROGRAM FOR INDIRECT MEDICAL IMAGE ANALYSIS

(71) Applicant: MEDYNUS INC., Irvine, CA (US)

(72) Inventors: Yongsuk CHO, Goyang-si (KR); Hyukchan KWON, Goyang-si (KR); Byunghui LIM, Seoul (KR)

(73) Assignee: MEDYNUS INC., Irvine, CA (US)

(21) Appl. No.: 18/987,923

(22) Filed: Dec. 19, 2024

(30) Foreign Application Priority Data
Oct. 30, 2024 (KR) 10-2024-0151076

(51) Int. Cl. G06T 7/00; G06T 5/60; G06T 5/70; G06T 7/60; G06T 7/73; G06V 10/764; G06V 10/94; G06V 20/70

(52) U.S. Cl. CPC G06T 7/0014; G06T 5/60; G06T 5/70; G06T 7/60; G06T 7/74; G06V 10/764; G06V 20/70; G06T 2207/10116; G06T 2207/20081; G06T 2207/30012; G06V 10/95; G06V 2201/033

(57) ABSTRACT

A method for indirect medical image analysis according to various embodiments of the present invention is disclosed. The method includes obtaining an indirect medical image captured from a medical image displayed on a monitor; and generating analysis information on the medical image by inputting the indirect medical image to a pre-trained image analysis model, wherein the image analysis model is pre-trained based on training data consisting of an original medical image and a noise medical image in which monitor noise is included in the original medical image.

### CROSS-REFERENCE TO RELATED APPLICATION

This application claims priority to and the benefit of Korean Patent Application No. 10-2024-0151076, filed on Oct. 30, 2024, the disclosure of which is incorporated herein by reference in its entirety.

### BACKGROUND

### 1. Field of the Invention

The present invention relates to a method, apparatus, and program for indirect medical image analysis, and more particularly, to a method, apparatus, and program for analyzing indirect medical images obtained by capturing images of a monitor on which original medical images are output.

### 2. Discussion of Related Art

Medical image analysis plays a very important role in modern medicine and is utilized in diagnosis and treatment plan establishment using various medical images obtained by x-rays, computerized tomography (CT) magnetic resonance imaging (MRI), etc. In particular, medical image analysis is essential for early diagnosis of a disease or evaluation of conditions before and after surgery through accurate reading and interpretation of medical images.

Most medical images today are managed through the Picture Archiving and Communication System (PACS), and medical staff check images through the corresponding system and use the images in diagnosis. However, the PACS mainly focuses on functions of storing and loading medical images and has a limitation in that an analysis function is insufficient. In particular, to analyze a medical image, the corresponding medical image should be extracted from the PACS to the outside, and various practical difficulties such as a connection problem between systems or compliance with personal information protection regulations occur. Due to such limitations, it is not easy to freely utilize images in the medical field.

In addition, a method of embedding analysis software (SW) in a medical device may also be considered, but this method comes with practical difficulties due to problems such as a limitation on device performance, complexity relating to system integration, and increases in maintenance and repair costs. When the SW is embedded in a medical device, a burden may be placed on a processing ability of the device, and various software updates and security management are also required.

Due to such practical limitations, demand for a method of indirectly capturing and analyzing medical images output on a monitor is present in the art. In relation to this, Korean Patent Registration No. 10-2063492 discloses “Method and System for Filtering Obstacle Data in Machine Learning of Medical Images.”

### SUMMARY OF THE INVENTION

The present invention is directed to providing a method, apparatus, and program for indirect medical image analysis.

The objects of the present invention are not limited to the above-mentioned object, and other unmentioned objects will be able to be clearly understood by those skilled in the art from the following description.

One embodiment of the present invention discloses a method for indirect medical image analysis. The method includes obtaining an indirect medical image captured from a medical image displayed on a monitor; and generating analysis information on the medical image by inputting the indirect medical image to a pre-trained image analysis model, wherein the image analysis model is pre-trained based on training data consisting of an original medical image and a noise medical image in which monitor noise is included in the original medical image.

In an alternative embodiment, the method may further include obtaining the original medical image; obtaining a monitor noise image by capturing an image of the monitor; and generating the noise medical image by synthesizing the original medical image and the monitor noise image.

In an alternative embodiment, the generating of the noise medical image by synthesizing the original medical image and the monitor noise image may include adjusting a brightness level of the original medical image; and obtaining the noise medical image by synthesizing the monitor noise image and the original medical image of which the brightness level is adjusted.

In an alternative embodiment, the generating of the noise medical image by synthesizing the original medical image and the monitor noise image may include when a plurality of monitor noise images are obtained, extracting a noise layer from each of the plurality of monitor noise images; recognizing a noise pattern included in each of the plurality of noise layers and classifying the plurality of noise layers by pattern; and generating a plurality of noise medical images by adjusting a brightness level of the original medical image and synthesizing each of the different noise patterns with the original medical image of which the brightness level is adjusted.

In an alternative embodiment, the method may further include detecting and labelling four vertices corresponding to each of a plurality of bones included in the images constituting the training data; and pre-training the image analysis model based on the training data on which the labelling is completed.

In an alternative embodiment, the image analysis model may recognize two points at an upper end among the four vertices corresponding to each of the plurality of bones as an upper line of each of the plurality of bones and may recognize two points at a lower end among the four vertices corresponding to each of the plurality of bones as a lower line of each of the plurality of bones.

In an alternative embodiment, the method may further include providing labelling information of the labelling of the four vertices corresponding to each of the plurality of bones; obtaining adjustment input information relating to the four vertices corresponding to each of the plurality of bones; and re-training the image analysis model after reflecting the adjustment input information in the training data.

In an alternative embodiment, when the indirect medical image is input thereto, the image analysis model may, detect at least two or more bones among the plurality of bones included in the indirect medical image, recognize distance and position relations between the at least two or more bones, and output the distance and position relations between the bones.

One embodiment of the present invention discloses an apparatus. The apparatus includes a memory storing one or more instructions; and a processor executing the one or more instructions stored in the memory, wherein the processor performs the above-described method by executing the one or more instructions.

One embodiment of the present invention discloses a computer-readable recording medium. The computer-readable recording medium may provide a surgery simulation method in combination with a computer which is hardware.

Other details of the present invention are incorporated in the detailed description and the drawings.

### BRIEF DESCRIPTION OF THE DRAWINGS

The above and other objects, features, and advantages of the present invention will become more apparent to those of ordinary skill in the art by describing exemplary embodiments thereof in detail with reference to the accompanying drawings, in which:

FIG. 1 is a view illustrating a system according to one embodiment of the present invention;

FIG. 2 is a hardware block diagram of a computing apparatus according to one embodiment of the present invention; and

FIGS. 3 to 7 are views for describing a method for indirect medical image analysis according to one embodiment of the present invention.

### DETAILED DESCRIPTION OF EXEMPLARY EMBODIMENTS

Various embodiments will be described below with reference to the accompanying drawings. In the present specification, various details are set forth to provide an understanding of the present invention. However, it is apparent that the embodiments may be practiced without these specific details.

The terms, “component,” “module,” “system,” and the like used herein indicate a computer-related entity, hardware, firmware, software, a combination of software and hardware, or execution of software. For example, a component may be a procedure executed in a processor, a processor, an object, an execution thread, a program, and/or a computer, but is not limited thereto. For example, both an application executed in a computing device and a computing device may be components. One or more components may reside within a processor and/or an execution thread. One component may be localized within one computer. One component may be distributed between two or more computers. Further, the components may be executed by various computer readable media having various data structures stored therein. For example, components may communicate through local and/or remote processing according to a signal (for example, data transmitted to another system through a network, such as the Internet, through data and/or a signal from one component interacting with another component in a local system and a distributed system) having one or more data packets.

In addition, the term “or” is intended to mean inclusive “or” instead of exclusive “or.” That is, unless specified otherwise or the context clearly indicates otherwise, “X uses A or B” is intended to mean one of natural inclusive substitutions. That is, “X uses A or B” may apply to any of the cases in which X uses A, X uses B, or X uses both A and B. Also, the term “and/or” used herein should be understood as referring to and including all possible combinations of one or more of the listed related items.

Also, the terms “include” and/or “including” should be understood as indicating the presence of corresponding features and/or components. However, the terms “include” and/or “including” should not be understood as excluding the presence or addition of one or more other features, components, and/or groups thereof. Also, unless specified otherwise or the context clearly indicates singularity, a singular expression should be generally interpreted as indicating “one or more” in the present specification and the claims.

Those skilled in the art should also recognize that various illustrative logical blocks, configurations, modules, circuits, means, logic, and algorithm operations described in relation to embodiments disclosed herein may be implemented by electronic hardware, computer software, or in a combination of electronic hardware and computer software. In order to clearly exemplify interchangeability of hardware and software, various illustrative components, blocks, configurations, means, logic, modules, circuits, and operations have been generally described above in the functional aspects thereof. Whether the functionality is implemented as hardware or software depends on the specific application or design restraints given to the overall system. Those skilled in the art may implement functionality described using various methods for each of specific applications. However, such decisions of implementations should not be construed as deviating from the scope of the present invention.

The description of embodiments set forth herein is provided to allow those of ordinary skill in the art to use or carry out the present invention. Various modifications of the embodiments should be apparent to those of ordinary skill in the art. General principles defined herein may also apply to other embodiments without departing from the scope of the present invention. Thus, the present invention is not limited to the embodiments set forth herein. The present invention should be construed in the widest possible sense consistent with principles and novel features set forth herein.

In the present specification, a computer is any type of hardware device including at least one processor and may be understood as encompassing software elements operating in the corresponding hardware device according to embodiments. For example, examples of a computer may be understood as including but not limited to all of a smartphone, a tablet personal computer (PC), a desktop, a laptop, and a user client and applications running on each of the devices.

Hereinafter, embodiments of the present invention will be described in detail with reference to the accompanying drawings.

Each step described herein is described as being performed by a computer, but the subject of each step is not limited thereto, and at least some of the steps may be performed in different devices according to embodiments.

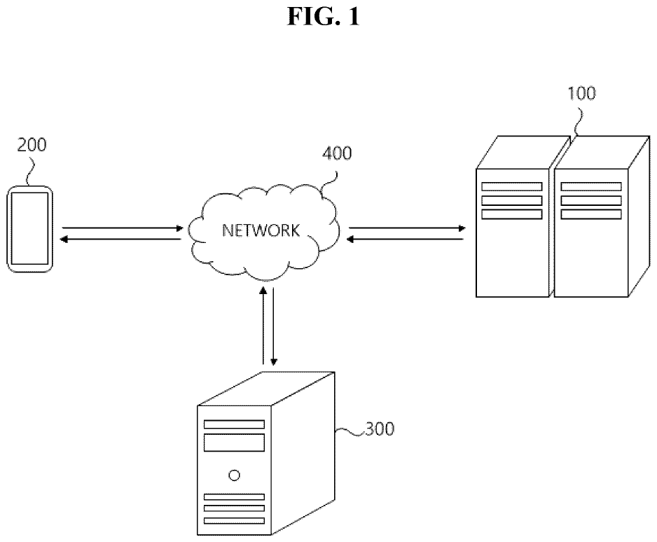

FIG. 1 is a view illustrating a system according to one embodiment of the present invention.

Referring to FIG. 1, the system according to one embodiment of the present invention may include a computing apparatus 100, a user terminal 200, and an external server 300. The system illustrated in FIG. 1 is only one embodiment, and components thereof are not limited to the embodiment illustrated in FIG. 1 and may be added, changed, or omitted as necessary.

In one embodiment, the computing apparatus 100 may perform a method for indirect medical image analysis. For example, the computing apparatus 100 may analyze an indirect medical image obtained by capturing an x-ray image output on a monitor, may analyze position information and distances of bones included in the x-ray image, and may generate analysis information based on the same.

In the present invention, an indirect medical image is an image obtained by a user or a device capturing an image of a monitor on which a medical image is output and may be an image indirectly obtained through a screen of the monitor instead of an original medical image directly extracted from the Picture Archiving and Communication System (PACS).

Specifically, the computing apparatus 100 may obtain an indirect medical image by capturing a medical image displayed on a monitor. In addition, the computing apparatus 100 may generate analysis information on the medical image by inputting the indirect medical image into a pre-trained image analysis model.

In one embodiment, the image analysis model may be pre-trained based on training data consisting of an original medical image and a noise medical image in which monitor noise is included in the original medical image.

In various embodiments, the image analysis model may be pre-trained based on training data consisting of an original medical image, a noise medical image in which monitor noise is included in the original medical image, and a noise-removed medical image in which the monitor noise is removed from the noise medical image.

In addition, the trained image analysis model may obtain an indirect medical image provided by the computing apparatus 100 as an input and may output analysis data such as distance and position information of bones. In this case, the computing apparatus 100 may generate analysis information based on the analysis data output by the model and may provide the analysis information to a user.

Accordingly, the computing apparatus 100 of the present invention may analyze geometrical information of bones through the indirect image obtained by capturing the medical image displayed on the monitor and may provide analysis information necessary for diagnosis.

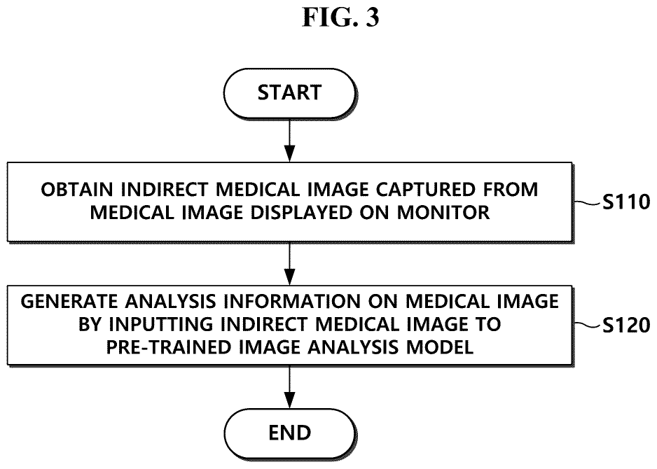

One example in which the computing apparatus 100 performs the method for indirect medical image analysis will be described below with reference to FIGS. 3 to 7.

In one embodiment, the computing apparatus 100 may obtain an analysis target image from the user terminal 200. Here, the analysis target image may be an indirect medical image captured by the user terminal 200. For example, the computing apparatus 100 may receive an analysis target image captured by the user terminal 200 through an application for indirect image analysis. That is, a user may capture a medical image displayed on a monitor using the user terminal 200 such as a smartphone or a tablet and then may send the corresponding image to the computing apparatus 100 through the application.

In this case, the computing apparatus 100 may obtain an analysis result by inputting the analysis target image into the image analysis model. In addition, the computing apparatus 100 may provide analysis information corresponding to the analysis result to the user terminal. For example, the computing apparatus 100 may provide the analysis information to the user terminal 200 through the application for indirect image analysis. In this way, the user may check the analysis result in real time within the application.

Accordingly, by allowing a user (e.g., medical staff) to conveniently check an analysis result using a smartphone without directly extracting data from the PACS, access to and efficiency of medical image analysis can be improved.

In various embodiments, the computing apparatus 100 may provide a web- or application-based service. However, the present invention is not limited thereto.

Examples of the computing apparatus 100 may include any type of computer system or computer device such as a microprocessor, a main frame computer, a digital processor, a portable device, and a device controller. However, the present invention is not limited thereto.

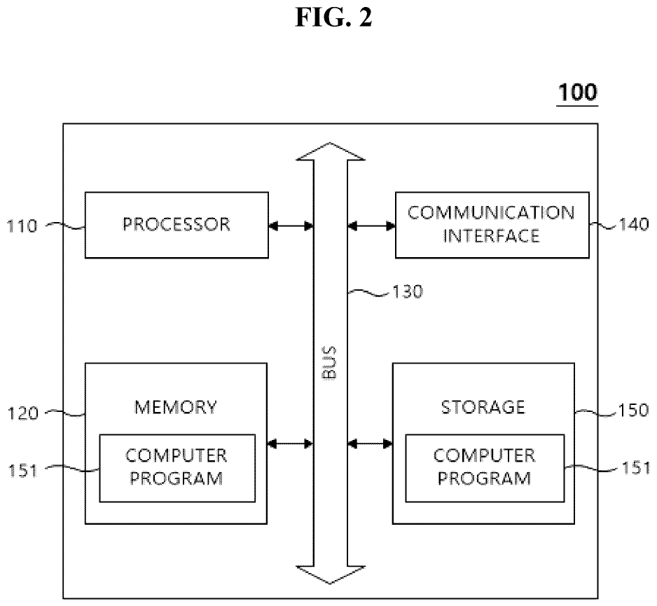

A hardware configuration of the computing apparatus 100 will be described below with reference to FIG. 2.

Meanwhile, the user terminal 200 may be connected to the computing apparatus 100 through a network 400 and may be a terminal of a user (e.g., a doctor or a patient) receiving analysis information on a medical image provided by the computing apparatus 100.

Here, examples of the user terminal 200 may include various forms of computing apparatuses. Specifically, for example, the user terminal 200 may be any of various terminal devices such as a smartphone, a tablet PC, a desktop, and a laptop.

The user terminal 200 may include a display on at least a portion thereof and may include an operating system for running a service based on an application or extension program provided by the computing apparatus 100. For example, the user terminal 200 may be a smartphone but is not limited thereto, and examples of the user terminal 200 may include, as a wireless communication device with ensured portability and mobility, any type of handheld-based wireless communication device such as navigation, Personal Communication System (PCS), Global System for Mobile communication (GSM), Personal Digital Cellular (PDC), Personal Handy-phone System (PHS), Personal Digital Assistant (PDA), International Mobile Telecommunication (IMT)-2000, Code Division Multiple Access (CDMA)-2000, Wideband Code Division Multiple Access (W-CDMA), a Wireless Broadband Internet (Wibro) terminal, a smart pad, and a tablet PC.

The external server 300 may be connected to the computing apparatus 100 through the network 400, may transmit and receive various information/data necessary for the computing apparatus 100 to perform the method for indirect medical image analysis, and may store and manage various information/data generated as the computing apparatus 100 performs the method for indirect medical image analysis.

For example, the external server 300 may be a database server storing information used in the method for indirect medical image analysis. As another example, the external server 300 may be a server providing information used in the method for indirect medical image analysis.

The network 400 may be a connection structure that enables information exchange between different nodes such as a computing apparatus, a plurality of terminals, and servers. Examples of the network 400 include a Local Area Network (LAN), a Wide Area Network (WAN), the World Wide Web (WWW), a wired/wireless data network, a telephone network, a wired/wireless television network, etc.

Examples of the wireless data network include a 3^{rd} Generation (3G) network, a 4^{th} Generation (4G) network, a 5^{th} Generation (5G) network, a 3^{rd} Generation Partnership Project (3GPP) network, a 5^{th} Generation Partnership Project (5GPP) network, a Long Term Evolution (LTE) network, a World Interoperability for Microwave Access (WIMAX) network, Wi-Fi, the Internet, a Local Area Network (LAN), a Wireless Local Area Network (WLAN), a Wide Area Network (WAN), a Personal Area Network (PAN), a Radio Frequency (RF) network, a Bluetooth network, a Near-Field Communication (NFC) network, a satellite broadcasting network, an analog broadcasting network, a Digital Multimedia Broadcasting (DMB) network, etc., but the present invention is not limited thereto.

FIG. 2 is a hardware block diagram of a computing apparatus according to one embodiment of the present invention.

Referring to FIG. 2, the computing apparatus 100 according to one embodiment of the present invention may include at least one processor 110, a memory 120 loading a computer program 151 performed by the processor 110, a bus 130, a communication interface 140, and a storage 150 storing the computer program 151. Here, only the components relating to the embodiment of the present invention are illustrated in FIG. 2. Accordingly, those of ordinary skill in the art to which the present invention pertains may recognize that the computing apparatus 100 may further include general-purpose components other than the components illustrated in FIG. 2.

The processor 110 controls the overall operation of each component of the computing apparatus 100. The processor 110 may be configured using one or more cores and may include a processor for data analysis or deep learning such as a central processing unit (CPU), a general-purpose graphics processing unit (GPGPU), or a tensor processing unit (TPU) of a computing apparatus. Alternatively, the processor 110 may be configured to include any other form of processor well known in the art.

In addition, the processor 110 may perform an arithmetic operation relating to at least one application or program for performing methods according to embodiments of the present invention, and the computing apparatus 100 may include one or more processors.

In various embodiments, the processor 110 may further include a random access memory (RAM) (not illustrated) and a read-only memory (ROM) (not illustrated) temporarily and/or permanently storing signals (or data) processed in the processor 110. In addition, the processor 110 may be implemented in the form of a system-on-chip (SoC) including at least one of a graphics processor, a RAM, and a ROM.

The memory 120 stores various data, commands, and/or information. The memory 120 may load the computer program 151 from the storage 150 to execute methods/operations according to various embodiments of the present invention. When the computer program 151 is loaded to the memory 120, the processor 110 may execute one or more instructions constituting the computer program 151 to perform the methods/operations. Although the memory 120 may be implemented using a volatile memory such as a RAM, the technical scope of the present invention is not limited thereto.

The bus 130 provides a communication function between the components of the computing apparatus 100. The bus 130 may be implemented in various forms of buses such as an address bus, a data bus, and a control bus.

The communication interface 140 supports wired/wireless Internet communication of the computing apparatus 100. In addition, the communication interface 140 may also support various communication methods other than the Internet communication. To this end, the communication interface 140 may be configured to include a communication module well known in the art. In some embodiments, the communication interface 140 may be omitted.

The storage 150 may non-provisionally store the computer program 151. When a process according to an embodiment of the present invention is performed through the computing apparatus 100, the storage 150 may store various information necessary for performing methods or providing services according to disclosed embodiments.

The storage 150 may be configured to include a nonvolatile memory such as a ROM, an erasable programmable ROM (EPROM), an electrically erasable programmable ROM (EEPROM), and a flash memory, a hard disk, a detachable disk, or a computer readable recording medium in an arbitrary form, which is well known in the art to which the present invention pertains.

The computer program 151 may include one or more instructions that allow the processor 110 to perform methods/operations according to various embodiments of the present invention when the computer program 151 is loaded to the memory 120. That is, by executing the one or more instructions, the processor 110 may perform the methods/operations according to various embodiments of the present invention.

In one embodiment, the computer program 151 may include one or more instructions that allow various methods related to various tasks related to learning of a neural network model to be performed.

The steps of a method or an algorithm that have been described in relation to the embodiments of the present invention may be directly implemented by hardware, may be implemented by a software module executed by hardware, or may be implemented by a combination thereof. The software module may reside in a RAM, a ROM, an EPROM, an EEPROM, a flash memory, a hard disk, a detachable disk, a CD-ROM, or a computer readable recording medium in an arbitrary form, which is well known in the art to which the present invention pertains.

The components of the present invention can be implemented as a program (or application) to be executed by being combined with a computer which is hardware and can be stored in a medium. The components of the present invention may be executed using software programming or software elements, and similarly, the embodiments may be implemented with any programming or scripting language such as C, C++, Java, and assembler, with the various algorithms being implemented with any combination of data structures, processes, routines, or other programming elements. Functional aspects may be implemented in algorithms that are executed on one or more processors.

FIGS. 3 to 7 are views for describing a method for indirect medical image analysis according to one embodiment of the present invention.

Referring to FIG. 3, the computing apparatus 100 may obtain an indirect medical image of a medical image displayed on a monitor (S110).

For example, the computing apparatus 100 may receive an indirect image, which is obtained by a user capturing a medical image displayed on a monitor, from the user terminal 200. Here, the user may send the captured image to the computing apparatus 100 through a mobile application installed in the user terminal 200.

As another example, the computing apparatus 100 may directly obtain an indirect medical image from a separate camera device installed to capture an image of a monitor. The camera device may be installed to be fixed to a monitor of a surgery room or an examining room and may be configured to capture an image in real time.

However, the present invention is not limited to the above-described examples, and the computing apparatus 100 may obtain an indirect medical image from various other sources such as the external server 300 connected through the network 400, a cloud storage, or another medical imaging device.

The computing apparatus 100 may generate analysis information on the medical image by inputting the indirect medical image to a pre-trained image analysis model (S120). Here, the image analysis model may analyze position information, distances, angles, etc. of bones included in the indirect medical image and may provide diagnosis assistance information necessary for medical staff.

In one embodiment, the image analysis model may be pre-trained based on training data consisting of an original medical image and a noise medical image in which monitor noise is included or training data consisting of an original medical image, a noise medical image in which monitor noise is included, and a noise-removed medical image. Here, the original medical image is a high-quality medical image and may be obtained from the PACS or the external server 300. The noise medical image is an image synthesized to include monitor noise or an image of a monitor on which the original medical image is output, and the noise-removed medical image may be an image obtained by removing noise from the noise medical image. The medical image analysis model may be trained by utilizing various training data listed above and may output an accurate result even when noise or distortion occurs due to capturing the image of the monitor.

For example, the computing apparatus 100 may pre-train the image analysis model by configuring two different types of training data, the original medical image and the noise medical image in which monitor noise is included, at a ratio of 1:1.

As another example, the computing apparatus 100 may pre-train the image analysis model by configuring three different types of training data, the original medical image, the noise medical image in which monitor noise is included, and the noise-removed medical image, at a ratio of 1:1:1.

In this way, the model can perform generalized learning on various image qualities and noise conditions and can provide highly accurate analysis results in a real environment.

In one embodiment, the image analysis model may be a model performing an integrated function of outputting an analysis result based on noise removal and a noise-removed image. In this case, the computing apparatus 100 may simultaneously perform noise removal and analysis by inputting an indirect medical image including noise into a single model. The integrated model may be trained using the noise-including image itself and may process noise removal and analysis at one time.

In various embodiments, the image analysis model may be separately configured into a noise removal model and an analysis model outputting an analysis result based on a noise-removed image. For example, the noise removal model may perform a role of removing noise from an indirect medical image, and then may deliver a noise-removed image to the analysis model to derive diagnosis assistance information such as the positions, angles, distances, etc. of bones. In such a configuration, each model may independently operate and be sequentially processed.

In various embodiments, the computing apparatus 100 may generate a noise medical image used in learning of the image analysis model. That is, the computing apparatus 100 may augment training data by simulating a noise pattern actually generated when capturing an image of the monitor.

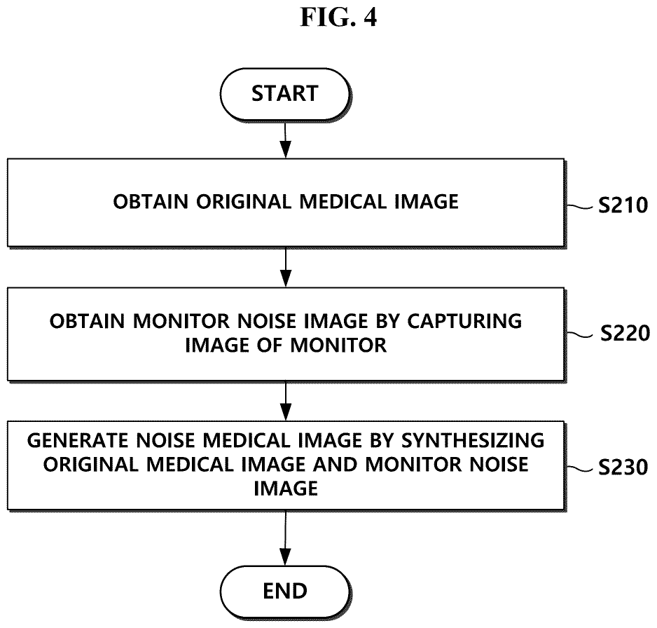

Specifically, referring to FIG. 4, the computing apparatus 100 may obtain an original medical image (S210).

For example, the computing apparatus 100 may obtain the original medical image by directly receiving it from the PACS or the external server 300. In addition, the computing apparatus 100 may obtain a monitor noise image through a camera of the user terminal 200 or a separate camera device installed on the monitor.

In addition, the computing apparatus 100 may obtain a monitor noise image by capturing an image of a monitor (S220).

For example, the computing apparatus 100 may obtain a monitor noise image in various environments through the camera of the user terminal 200 or the separate camera device installed on the monitor. Here, the monitor noise image is obtained by capturing images of monitors of various sizes and resolutions at various angles and under various lighting conditions, and for example, the monitor noise image may include various types of noise actually generated during image capturing such as a Moié pattern, a reflection, or screen noise.

In one embodiment, the computing apparatus 100 may generate a noise medical image by synthesizing the original medical image and the monitor noise image when the original medical image and the monitor noise image are obtained (S230).

For example, the computing apparatus 100 may generate a noise medical image having noise characteristics similar to when actually capturing an image of the monitor and may train the model to be able to respond to various noise situations.

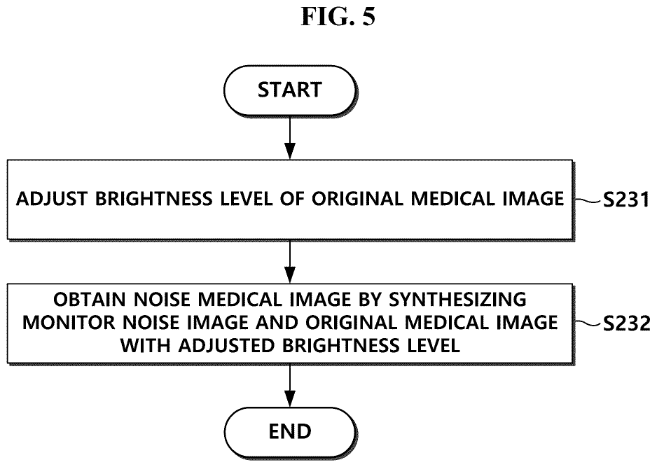

Specifically, referring to FIG. 5, the computing apparatus 100 may adjust a brightness level of an original medical image (S231).

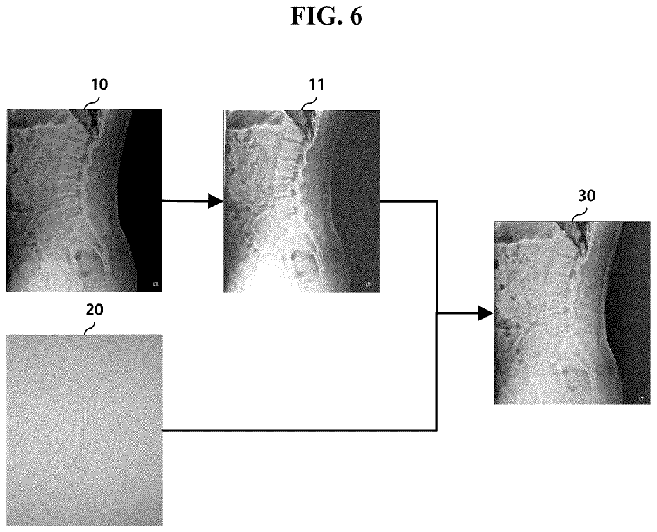

For example, the computing apparatus 100 may apply a brightness adjustment algorithm to the original medical image to improve a contrast of the image. Specifically, for example, the computing apparatus 100 may apply gamma correction or histogram equalization to the original medical image. In this way, the computing apparatus 100 may allow important features such as the structures of bones to be more clearly shown. For example, as illustrated in FIG. 6, the computing apparatus 100 may adjust a brightness level of an original medical image 10 to improve a contrast thereof and may generate an original medical image 11 with an adjusted brightness level.

In addition, the computing apparatus 100 may obtain a noise medical image by synthesizing a monitor noise image and the original medical image with an adjusted brightness level (S232).

Specifically, the computing apparatus 100 may adjust each of the monitor noise image and the original medical image with an adjusted brightness level to the same resolution and size. In addition, the computing apparatus 100 may adjust the transparency (alpha value) of the monitor noise image and may overlap the monitor noise image on the original medical image with an adjusted brightness level. Here, the transparency of the monitor noise image may be set to a value between 0.1 and 0.5 to reflect the strength of noise actually generated when capturing an image of the monitor.

For example, the computing apparatus 100 may generate a noise medical image by setting the transparency of the monitor noise image as 0.3 and synthesizing the original medical image with an adjusted brightness level and the monitor noise image in pixels. For example, as illustrated in FIG. 6, the computing apparatus 100 may generate a noise medical image 30 by synthesizing the original medical image 11 with an adjusted brightness level and a monitor noise image 20 in pixels.

In addition, the computing apparatus 100 may generate various forms of noise medical images through different combinations of the position, size, rotation, transformation, and the like of the monitor noise image in the synthesizing process. Through such data augmentation, the computing apparatus 100 may train the model to show more diverse noise conditions and have high performance for image distortion.

In various embodiments, the computing apparatus 100 may extract a noise layer by removing a background from the monitor noise image.

For example, the computing apparatus 100 may apply an image segmentation technique or color thresholding to the monitor noise image to remove a background region and extract only a noise pattern. In this way, the computing apparatus 100 may generate a pure noise layer that may be synthesized with the original medical image.

In addition, the computing apparatus 100 may obtain a noise medical image by synthesizing the original medical image with an adjusted brightness level and the noise layer.

Specifically, the computing apparatus 100 may adjust each of the original medical image with an adjusted brightness level and the noise layer to the same resolution and size. In addition, the computing apparatus 100 may adjust the transparency (alpha value) of the noise layer and may overlap the noise layer on the original medical image with an adjusted brightness level. Here, the transparency of the noise layer may be set to a value between 0.1 and 0.5 to reflect the strength of noise actually generated when capturing an image of the monitor.

For example, the computing apparatus 100 may generate a noise medical image by setting the transparency of the noise layer as 0.3 and synthesizing the original medical image with an adjusted brightness level and the noise layer in pixels.

In addition, the computing apparatus 100 may generate various forms of noise medical images through different combinations of the position, size, rotation, transformation, and the like of the noise layer in the synthesizing process. Through such data augmentation, the computing apparatus 100 may train the model to show more diverse noise conditions and have high performance for image distortion.

In various embodiments, the computing apparatus 100 may generate not only the noise medical image but also a noise-removed medical image by removing noise from the noise medical image.

Specifically, the computing apparatus 100 may obtain a noise-removed medical image by applying a noise removal filter, such as a Gaussian blur or a median filter, to the noise medical image or utilizing a deep learning-based noise removal algorithm such as an autoencoder.

In addition, the computing apparatus 100 may obtain a noise-removed medical image through a pre-trained image analysis model for noise removal. For example, a deep learning model specialized for noise removal may be trained separately, and the corresponding model may be utilized to remove noise from the noise medical image.

The computing apparatus 100 may pre-train an image analysis model by utilizing various training data generated as described above. The trained image analysis model obtains an indirect medical image provided by the computing apparatus 100 as an input and outputs analysis data such as distance and position information of bones. The computing apparatus 100 may generate analysis information, including the structural characteristics, alignment states, deformation, and the like of bones, based on the analysis data output by the model and may provide the analysis information to a user.

Accordingly, the computing apparatus 100 of the present invention can provide accurate and reliable diagnosis assistance information to medical staff by effectively analyzing an indirect medical image of a medical image displayed on a monitor. In this way, the present invention can improve diagnosis efficiency in the medical field and can provide a high-quality medical image analysis function without the PACS or complex linkage with a medical device.

According to various embodiments of the present invention, in the generating of the noise medical image by synthesizing the original medical image and the monitor noise image (S120), a plurality of noise medical images may be generated.

Specifically, when a plurality of monitor noise images are obtained, the computing apparatus 100 may extract a noise layer from each of the plurality of monitor noise images. Here, the monitor noise images may be captured under various monitor type, resolution, brightness, color temperature, capturing angle, and lighting conditions and may include various noise patterns. For example, the noise patterns may be classified into a Moié pattern, a screen dot, reflection light noise, pixel noise, etc.

The computing apparatus 100 may recognize a noise pattern included in each of the plurality of noise layers and may classify the plurality of noise layers by pattern.

Specifically, the computing apparatus 100 may recognize a noise pattern through frequency analysis (the Fourier transform) and an image processing algorithm and may classify the plurality of noise layers by pattern. For example, the computing apparatus 100 may detect a periodic Moié pattern through frequency analysis or may recognize screen dots or pixel noise using an image filtering technique.

More specifically, the computing apparatus 100 may analyze frequency components of the monitor noise images and may classify the frequency components into high-frequency noise, low-frequency noise, and periodic pattern noise. In addition, the computing apparatus 100 may recognize a repeated pattern, such as screen dots, by applying an edge detection algorithm and may measure a strength of reflection light noise through histogram analysis. For example, the computing apparatus 100 may detect a Moié pattern by analyzing a frequency spectrum and may classify a pattern such as screen dots using the image filtering technique. In addition, the computing apparatus 100 may recognize reflection light through optical flow analysis and may classify pixel noise based on a change in pixel strength.

The computing apparatus 100 may generate a plurality of noise medical images by adjusting a brightness level of the original medical image and synthesizing each of the different noise patterns with the original medical image of which the brightness level is adjusted.

Specifically, the computing apparatus 100 may adjust a brightness level by applying gamma correction or histogram equalization to the original medical image. In this way, the computing apparatus 100 may improve a contrast of the image and emphasize important details (e.g., bone parts) to maintain the quality of the original image at the time of noise synthesis.

In addition, the computing apparatus 100 may synthesize the classified different noise patterns (e.g., a Moié pattern, screen dots, reflection light, pixel noise) with the original medical image with an adjusted brightness level and may generate a plurality of noise medical images to which the different noise patterns are applied.

For example, the computing apparatus 100 may generate training data reflecting various noise conditions by generating, using a single brightness-corrected original medical image, each of an image in which Moié pattern noise is synthesized, an image in which screen dot noise is synthesized, an image in which reflection light is included, and an image in which pixel noise is added.

Accordingly, the computing apparatus 100 may generate training data for the image analysis model to have high performance for noise and distortion that may occur in various environments.

In addition, the computing apparatus 100 may synthesize each noise layer with a brightness-adjusted original medical image through transparency (alpha value) control. Here, the transparency of each noise layer may be set to a value between 0.1 and 0.5 to reflect the strength of noise actually generated when capturing an image of the monitor.

In addition, the computing apparatus 100 may obtain various synthesis results by adjusting the position, size, rotation, distortion, and the like of each noise layer. For example, a Moié pattern may be rotated at a specific angle for synthesis, screen dots may have their sizes adjusted for synthesis, and reflection light may be distorted to be focused on a specific position for synthesis.

Accordingly, the computing apparatus 100 may generate training data for the image analysis model to have high performance for more diverse types of noise and environments by generating noise medical images that not only reflect various noise patterns but also include noise patterns deformed in various ways through different combinations of the position, strength, size, and deformation of each type of noise.

According to various embodiments of the present invention, the computing apparatus 100 may pre-train the image analysis model by performing labelling on the training data.

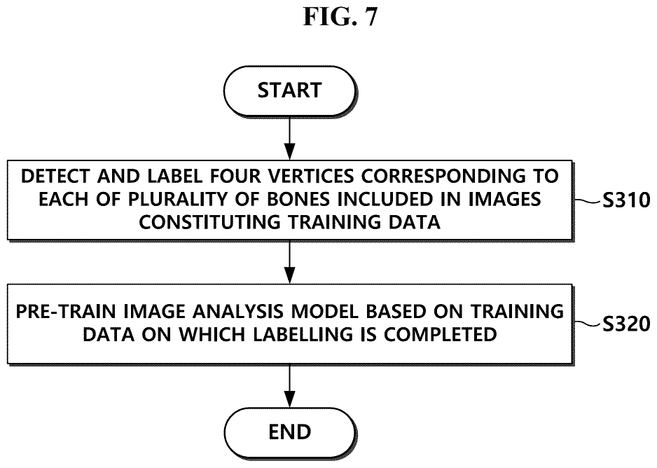

Referring to FIG. 7, the computing apparatus 100 may detect and label four vertices corresponding to each of a plurality of bones included in images constituting training data (S310). In addition, the computing apparatus 100 may pre-train an image analysis model based on the training data on which the labelling is completed (S320).

Specifically, the computing apparatus 100 may detect vertices of each bone by utilizing an object detection model such as You Only Live Once (YOLO), U-Net, Faster R-CNN, and Single Shot MultiBox Detector (SSD). In addition, the computing apparatus 100 may label the detected vertices on images to constitute training data.

Meanwhile, the computing apparatus 100 may pre-train the image analysis model using the training data on which the labelling is completed. In this case, the image analysis model may learn information on each vertex of bone, may detect the position of the bone from an indirect medical image afterwards, and may recognize an upper line and a lower line of the bone. Specifically, the image analysis model may recognize two points at an upper end among the four vertices corresponding to each of the plurality of bones as an upper line of each of the plurality of bones. In addition, the image analysis model may recognize two points at a lower end among the four vertices corresponding to each of the plurality of bones as a lower line of each of the plurality of bones.

In various embodiments, when vertices of each bone included in an image are recognized, the computing apparatus 100 may label the vertices corresponding to each bone with identification information.

For example, in the case of an image of the spine, the computing apparatus 100 may label each of vertices corresponding to the bones of the cervical spine with identification information (e.g., C1 to C7). In addition, the computing apparatus 100 may label each of vertices corresponding to the bones of the thoracic spine with identification information (e.g., T1 to T12). In addition, the computing apparatus 100 may label each of vertices corresponding to the bones of the lumbar spine with identification information (e.g., L1 to L5).

In various embodiments, the computing apparatus 100 may further label two points at an upper end (that is, the upper left point and the upper right point) among the four vertices corresponding to each of the plurality of bones as an upper line. In addition, the computing apparatus 100 may further label two points at a lower end (that is, the lower left point and the lower right point) among the four vertices corresponding to each of the plurality of bones as a lower line.

Specifically, the computing apparatus 100 may extract an upper end side and a lower end side using coordinate information of the vertices and may designate the upper end side and the lower end side as the upper line and the lower line, respectively. The labelling task relating to the upper line and the lower line may be performed in an automated manner by the computing apparatus 100 or may be manually adjusted through an interface by a user as necessary. For example, when an automatic labelling result is not accurate, a user may directly correct the positions of the vertices or the positions of the upper line and the lower line to improve accuracy.

Meanwhile, the computing apparatus 100 may pre-train the image analysis model using the training data on which the labelling is completed. In this case, the image analysis model may learn information on the positions of bones and the upper line and the lower line of each bone, may accurately detect the positions of the bones from an indirect medical image afterwards, and may recognize the upper line and the lower line of each bone. In this way, the present invention can analyze distances, angles, alignment states, deformation, and the like between bones and may provide useful information to medical staff.

In various embodiments, the computing apparatus 100 may recognize a boundary box of each of the plurality of bones included in the images constituting the training data. Here, the boundary box may be recognized in a rectangular or other polygonal form.

Specifically, the computing apparatus 100 may apply a deep learning-based object detection algorithm to detect the positions of bones in an image.

For example, the computing apparatus 100 may recognize a boundary box of each bone by utilizing an object detection model such as YOLO, U-Net, Faster R-CNN, and SSD. Such models may use pre-trained weights or may be re-trained through transfer learning according to characteristics of bones.

More specifically, the computing apparatus 100 may perform a preprocessing process (e.g., image resolution adjustment, noise removal, contrast improvement, etc.) for each image and then may input the preprocessed images to the object detection model and generate a boundary box showing the position and size of each bone. Here, the boundary box may be defined as a rectangular region of a minimum size that surrounds a bone or may be designated to have a polygonal shape to more accurately reflect the shape of the bone.

For example, the computing apparatus 100 may accurately segment regions of a bone by utilizing a segmentation model such as U-Net or Mask R-CNN. Here, the segmentation model determines whether each pixel in an image belongs to a bone and generates a mask in pixels. In addition, the segmentation model may extract an outline of the generated mask and may form a polygonal boundary box.

In various embodiments, when a boundary box of each bone included in an image is recognized, the computing apparatus 100 may label each bone with corresponding identification information.

For example, in the case of an image of the spine, the computing apparatus 100 may label each of the bones of the cervical spine with identification information (e.g., C1 to C7). In addition, the computing apparatus 100 may label each of the bones of the thoracic spine with identification information (e.g., T1 to T12). In addition, the computing apparatus 100 may label each of the bones of the lumbar spine with identification information (e.g., L1 to L5).

Next, the computing apparatus 100 that has recognized a boundary box may label an upper end of the boundary box as a line corresponding to an upper margin and may label a lower end of the boundary box as a line corresponding to a lower margin.

Specifically, the computing apparatus 100 may extract an upper end side and a lower end side using coordinate information of the boundary box and may designate the upper end side and the lower end side as the line corresponding to the upper margin and the line corresponding to the lower margin, respectively.

For example, in the case of a rectangular boundary box, the computing apparatus 100 may label a line connecting two coordinates constituting an upper corner (that is, an upper end of the boundary box) as the line corresponding to the upper margin and may label a line connecting two coordinates constituting a lower corner (that is, a lower end of the boundary box) as the line corresponding to the lower margin.

As another example, in the case of a polygonal boundary box, the computing apparatus 100 may label a line connecting the highest y-coordinates at a left side and a right side with respect to a central axis of the boundary box as the line corresponding to the upper margin and may label a line connecting the lowest y-coordinates at the left side and the right side with respect to the central axis of the boundary box as the line corresponding to the lower margin. Here, the central axis of the boundary box is a vertical line reflecting the left-right symmetry of the boundary box surrounding the bone and may be a virtual line located in between a horizontal width of the left side and a horizontal width of the right side of the boundary box.

The labelling task relating to the lines corresponding to the upper margin and the lower margin may be performed in an automated manner by the computing apparatus 100 or may be manually adjusted through an interface by a user as necessary. For example, when an automatic labelling result is not accurate, a user may directly correct the positions of the upper margin and the lower margin to improve accuracy.

Meanwhile, the computing apparatus 100 may pre-train the image analysis model using the training data on which the labelling is completed. In this case, the image analysis model may learn information on the positions of bones and the upper margin and the lower margin of each bone, may accurately detect the positions of the bones from an indirect medical image afterwards, and may recognize the upper margin and the lower margin of each bone. In this way, the present invention can analyze distances, angles, alignment states, deformation, and the like between bones and may provide useful information to medical staff.

Meanwhile, the image analysis model that is pre-trained by the method described above with reference to FIG. 7 may, when an indirect medical image is input thereto, detect at least two or more bones among a plurality of bones included in the indirect medical image. In addition, the image analysis model may recognize distance and position relations between the at least two or more bones and may output the distance and position relations between the bones.

In this case, the computing apparatus 100 may generate analysis information based on the distance and position relations between the bones.

Specifically, the computing apparatus 100 may calculate the distances, angles, alignment states, and the like between bones using position coordinate and shape information of bones that is extracted through the image analysis model. Through the calculation, the computing apparatus 100 may analyze structural characteristics of the bones and determine deformation or abnormality of the bones and may generate analysis information.

For example, the computing apparatus 100 may generate analysis information including a lumbar lordosis (LL) angle by measuring an angle between a first lumbar upper margin and a first sacral upper margin using the position coordinate and shape information of bones.

In various embodiments, the pre-trained image analysis model may be trained to immediately output an analysis result based on a predefined analysis method.

For example, the image analysis model may output an analysis result corresponding to each parameter by simultaneously learning analysis methods relating to one or more of information on local parameters corresponding to a cervical lordosis angle, a thoracic kyphosis angle, and a lumbar lordosis (LL) angle, systemic parameters corresponding to the sagittal axis and a first thoracic-pelvic angle, spinal-pelvic parameters corresponding to a pelvic incidence angle, a pelvic tilt, and a sacral inclination angle, and coronal parameters corresponding to a Cobb angle and coronal balance.

Specifically, the image analysis model may automatically recognize structures of bones in an image and may calculate measurement points and angles necessary for each parameter.

For example, the image analysis model may measure an angle formed between two specific bones of the cervical spine (for example, a lower margin of the second cervical vertebra C2 and a lower margin of the seventh cervical vertebra C7) to calculate and output the cervical lordosis angle. Here, the cervical lordosis angle is used to evaluate a normal lordosis curvature of the cervical spine, and a deformation or alignment state of the cervical spine may be determined through this numerical value.

As another example, the image analysis model may measure an angle formed between two specific bones of the thoracic spine (for example, an upper margin of the first thoracic vertebra T1 and a lower margin of the twelfth thoracic vertebra T12) to calculate and output the thoracic kyphosis angle. Here, the thoracic kyphosis angle shows the degree of curvature of the thoracic spine and may be utilized in analyzing the overall alignment state of the spine and whether there is an abnormality such as kyphosis.

As still another example, the image analysis model may calculate the lumbar lordosis (LL) angle based on an upper margin of the first lumbar vertebra L1 and a lower margin of the fifth lumbar vertebra L5 constituting the lumbar spine and may output the lumbar lordosis (LL) angle. Here, the lumbar lordosis (LL) angle shows the lordosis curvature of the lumbar spine and may be used in evaluating structural abnormality or deformation of the lumbar spine.

In this way, the image analysis model may calculate various spinal parameters by calculating distances and angles between specific points of bones and may output the spinal parameters as analysis information. The analysis information may be usefully utilized by medical staff in accurately understanding the spine condition of a patient and establishing diagnosis and treatment plans.

In various embodiments, when the labelling on the training data is completed, the computing apparatus 100 may correct labelling based on adjustment input information relating to labels.

Specifically, the computing apparatus 100 may provide labelling information of the labelling of the four vertices corresponding to each of the plurality of bones in the training data.

For example, the computing apparatus 100 may provide a labelling state of each vertex of bones for each image of training data to a user through a web application or dedicated software. In this case, the user may adjust the position of a vertex through a mouse or a touch input on an interface or may add a new vertex as necessary. For example, when a specific vertex does not coincide with an actual vertex of a bone, the user may drag the specific vertex and move it to an accurate position.

The computing apparatus 100 may obtain adjustment input information relating to the four vertices corresponding to each of the plurality of bones. Here, the adjustment input information may include a form in which the user corrects the position of a labelled vertex or inputs an additional comment.

In this case, the computing apparatus 100 may re-train the image analysis model after reflecting the adjustment input information in the training data. For example, the computing apparatus 100 may receive the adjustment input information of the user, may update the existing labelling information, and may re-train the image analysis model using the training data that includes the corrected labelling information.

Therefore, the computing apparatus 100 may improve labelling accuracy by utilizing the adjustment input information of the user and may improve performance of the image analysis model based on the improved labelling accuracy.

In other various embodiments, when the labelling on the training data is completed, the computing apparatus 100 may correct labelling based on adjustment input information relating to labels.

Specifically, the computing apparatus 100 may provide labelling information of the labelling of the lines corresponding to an upper margin and a lower margin of each of the plurality of bones in the training data.

For example, the computing apparatus 100 may provide labelling states of lines corresponding to an upper margin and a lower margin of bones for each image of training data to a user through a web application or dedicated software. In this case, the user may adjust the positions of the upper margin and lower margin lines through a mouse or a touch input on an interface or may add a new line as necessary. For example, when a line does not coincide with an actual boundary of a bone, the user may drag the line and move it to an accurate position.

The computing apparatus 100 may obtain adjustment input information relating to the lines corresponding to an upper margin and a lower margin. Here, the adjustment input information may include a form in which the user corrects the position or angle of a labelled line or inputs an additional comment.

In this case, the computing apparatus 100 may re-train the image analysis model after reflecting the adjustment input information in the training data. For example, the computing apparatus 100 may receive the adjustment input information of the user, may update the existing labelling information, and may re-train the image analysis model using training data that includes the corrected labelling information.

Therefore, the computing apparatus 100 may improve labelling accuracy by utilizing the adjustment input information of the user and may improve performance of the image analysis model based on the improved labelling accuracy.

According to an additional embodiment of the present invention, the computing apparatus 100 may selectively use a medical image analysis model by comparing accuracy of a result of learning with a boundary box recognized as a quadrangular shape and accuracy of a result of learning with a boundary box recognized as a polygonal shape.

Specifically, the computing apparatus 100 may use a quadrangular boundary box-based model to analyze an indirect medical image and extract information such as the position, the upper margin, the lower margin, and the like of each bone. In addition, the computing apparatus 100 may use a polygonal boundary box-based model to analyze the same indirect medical image and extract the same information.

In addition, the computing apparatus 100 may compare analysis results of the two models with the actual labelling data or a reading result of a medical expert and may determine the accuracy of each model. For example, the computing apparatus 100 may determine the accuracy of each of the two models based on precision, recall, F1-score, Intersection over Union (IoU), and the like.

In addition, the computing apparatus 100 may perform medical image analysis by selecting a specific model having relatively higher accuracy.

Accordingly, the computing apparatus 100 can improve accuracy and efficiency of medical image analysis by selectively applying a model with high accuracy.

In an additional embodiment, the computing apparatus 100 may automate model selection by utilizing a machine learning algorithm. For example, the computing apparatus 100 may apply a meta-learning technique, may predict which model will be more suitable according to features of an input image, and may selectively apply a model as a result.

Specifically, the computing apparatus 100 may extract features such as a resolution, a noise level, bone form complexity, a contrast, and a distortion degree from an input image. In addition, the computing apparatus 100 may input feature values to a pre-trained meta-learning model and may predict performance of each of the quadrangular boundary box-based model and the polygonal boundary box-based model.

Here, the metal-learning model is a model pre-trained based on the relations between various image features and the performance of the two models on the corresponding features and may predict which model will exhibit better performance for a newly input image. In this way, the computing apparatus 100 may automatically select and apply an analysis model that is the most suitable for characteristics of an input image.

In this way, the computing apparatus 100 may improve accuracy and efficiency of medical image analysis and may construct a flexible analysis system that exhibits optimal performance under various conditions.

According to the present invention, by analyzing an indirect medical image of a medical image displayed on a monitor, geometrical characteristics such as position information and distances of bones can be recognized, and analysis information can be provided based on the same.

The effects of the present invention are not limited to the above-mentioned effects, and other unmentioned effects should be clearly understood by those skilled in the art from the above description.

Although embodiments of the present invention have been described above with reference to the accompanying drawings, those of ordinary skill in the art to which the present invention pertains should understand that the present invention may be carried out in other specific forms without changing the technical spirit or essential features thereof. Therefore, the embodiments described above should be understood as illustrative, instead of limiting, in all aspects.

What is claimed is:

1. A method for indirect medical image analysis that is performed by a computing apparatus including at least one processor, the method comprising:
obtaining an indirect medical image captured from a medical image displayed on a monitor; and
generating analysis information on the medical image by inputting the indirect medical image to a pre-trained image analysis model,
wherein the image analysis model is pre-trained based on training data consisting of an original medical image and a noise medical image in which monitor noise is included in the original medical image.

2. The method of claim 1, further comprising:
obtaining the original medical image;
obtaining a monitor noise image by capturing an image of the monitor; and
generating the noise medical image by synthesizing the original medical image and the monitor noise image

3. The method of claim 2, wherein the generating of the noise medical image by synthesizing the original medical image and the monitor noise image includes:
adjusting a brightness level of the original medical image; and
obtaining the noise medical image by synthesizing the monitor noise image and the original medical image of which the brightness level is adjusted.

4. The method of claim 2, wherein the generating of the noise medical image by synthesizing the original medical image and the monitor noise image includes:
when a plurality of monitor noise images are obtained, extracting a noise layer from each of the plurality of monitor noise images;
recognizing a noise pattern included in each of the plurality of noise layers and classifying the plurality of noise layers by pattern; and
generating a plurality of noise medical images by adjusting a brightness level of the original medical image and synthesizing each of the different noise patterns with the original medical image of which the brightness level is adjusted.

5. The method of claim 1, further comprising:
detecting and labelling four vertices corresponding to each of a plurality of bones included in the images constituting the training data; and
pre-training the image analysis model based on the training data on which the labelling is completed.

6. The method of claim 5, wherein the image analysis model:
recognizes two points at an upper end among the four vertices corresponding to each of the plurality of bones as an upper line of each of the plurality of bones; and
recognizes two points at a lower end among the four vertices corresponding to each of the plurality of bones as a lower line of each of the plurality of bones.

7. The method of claim 5, further comprising:
providing labelling information of the labelling of the four vertices corresponding to each of the plurality of bones;
obtaining adjustment input information relating to the four vertices corresponding to each of the plurality of bones; and
re-training the image analysis model after reflecting the adjustment input information in the training data.

8. The method of claim 1, wherein, when the indirect medical image is input thereto, the image analysis model:
detects at least two or more bones among the plurality of bones included in the indirect medical image;
recognizes distance and position relations between the at least two or more bones; and
outputs the distance and position relations between the bones.

9. An apparatus comprising:
a memory storing one or more instructions; and
a processor executing the one or more instructions stored in the memory,
wherein the processor performs the method of claim 1 by executing the one or more instructions.

10. A computer-readable recording medium on which a program for executing a method of indirect medical image analysis with a computing device is recorded, wherein the method comprises:
obtaining an indirect medical image captured from a medical image displayed on a monitor; and
generating analysis information on the medical image by inputting the indirect medical image to a pre-trained image analysis model,
wherein the image analysis model is pre-trained based on training data consisting of an original medical image and a noise medical image in which monitor noise is included in the original medical image.
